(19) United States
(12) Patent Application Publication
(10) Pub. No.: US 2016/0000955 A1
(43) Pub. Date: Jan. 7, 2016

(54) Combined Odor Absorption and Insect Repellent Compositions, Treatments, and Methods of Use

(71) Applicant: Clean Control Corporation, Warner Robins, GA (US)

(72) Inventors: Stephen Davison, Perry, GA (US); Cory S. Hammock, Macon, GA (US)

(21) Appl. No.: 14/790,794

(22) Filed: Jul. 2, 2015

Related U.S. Application Data
(60) Provisional application No. 62/021,542, filed on Jul. 7, 2014.

(51) Int. Cl. A61L 9/01; A01N 43/40

(52) U.S. Cl. CPC A61L 9/01; A01N 43/40; A61L 2209/21

(57) ABSTRACT

Combined insect repellents and odor-reducing compositions are disclosed that use the insect repellent Picaridin [2-(2-hydroxyethyl)-1-piperidine carboxylic acid 1-methylpropyl ester] in combination with the odor-reducer zinc ricinoleate are disclosed, as are methods for using these compositions. The presence of the odor reducer does not reduce the apparent efficacy of the Picaridin, and tends to reduce the odor of the Picaridin itself.

### CROSS-REFERENCE TO RELATED APPLICATIONS

This application claims priority to U.S. Provisional Patent Application No. 62/021,542, filed Jul. 7, 2014. The contents of that application are incorporated by reference in their entirety.

### BACKGROUND OF THE INVENTION

1. Field of the Invention

The invention relates to odor absorption and insect repellent compositions.

2. Description of Related Art

DEET (N,N-diethyl-3-methylbenzamide) and Picaridin [2-(2-hydroxyethyl)-1-piperidine carboxylic acid 1-methylpropyl ester] offer protection against the bites of blood-feeding arthropods that vector human disease. Despite the widespread knowledge of the protective qualities of these compounds, as measured by reduced bites sustained in field and laboratory tests, there is little information available on how the compounds mechanistically affect whole-organism behavior and thereby suppress the biting of insects. Information in this area is particularly scarce for Picaridin. However, for DEET, which was discovered decades before Picaridin, there is a slightly larger bank of information.

Schreck et al. (1970) studied the action of DEET and candidate mosquito repellents. They observed that “mosquitoes exposed to an arm treated with repellent approach but do not land, land momentarily, land and walk, probe, and bite, in that order, as the amount of repellent is reduced, either artificially or by aging. Therefore, repellents do have a spatial effect that contributes to preventing insect attack.” They went on to say, “none of the repellents completely prevented all mosquitoes from responding to the test subjects (humans). Therefore, repellents may initially reduce annoyance by preventing close approach of a relatively high proportion of avid mosquitoes, but complete protection from biting depends on both spatial and contact repellency.”

Boeckh et al. (1996) studied the protective efficacy of DEET and Picaridin against yellow fever mosquito, and showed that Picaridin protected guinea pigs from bites for longer times, post application, than DEET. Reeder et al. (2001) concluded that DEET repellent effect in fruit flies was because of its airborne vapors. Hoffmann and Miller (2002, 2003) also showed that DEET has an olfactory effect and evaporated upwind of attractant-baited traps or human subjects reduced mosquito upwind orientation to an attractive source compared with moving air without the compound. Carroll et al. (2005) showed that the repellent activity of DEET against ticks involved olfactory sensing. Klun et al. (2006) concluded that DEET and Picaridin exert two behavioral effects on the insects. One is a feeding deterrent effect and the other is a repellent effect. Both effects are expressed as result of insect olfactory perception of the compounds.

Because of their properties, repellents like DEET and Picaridin are often used during outdoor activities, like camping, hiking, and hunting, either by spraying on clothes or on skin. However, these compounds have distinctive odors that are discernible to humans and animals.

Zinc ricinoleate (the zinc salt of 12-hydroxy-9-cis-octadecenoic acid) is a well-known odor-reducing compound that acts by adsorbing or sequestering odor-active compounds, and is particularly effective with nitrogen and sulfur compounds of relatively low molecular weight. While zinc ricinoleate is common in detergent and surfactant compositions, U.S. patent application Ser. No. 13/360,369, filed Jan. 27, 2012, the work of the present applicant which is incorporated by reference in its entirety, discloses the use of zinc ricinoleate as the primary active ingredient in odor-reducing solutions. These solutions are sold for use in hunting, to remove or reduce human scent, by Clean Control Corporation (Warner Robins, Ga., United States) under the brand name LETHAL®.

### SUMMARY OF THE INVENTION

One aspect of the invention relates to a method for creating an insect repellent effect with reduced odor characteristics. The method comprises applying the insect repellent Picaridin and also applying the odor-reducer zinc ricinoleate. One application may be permitted to dry before the other is applied. Typically, the two compounds would be applied in solution or suspension form, although other forms may be used.

Other aspects of the invention relate to kits that include Picaridin and zinc ricinoleate, as well as instructions for applying the two compounds together. Methods, kits and solutions according to aspects of the invention may be particularly useful in outdoor activities like hunting, where it is desirable to have an insect repellent while also eliminating or masking both the odor of the repellent and human odor.

Other aspects, features, and advantages of the invention will be set forth in the description that follows.

### DETAILED DESCRIPTION

The present inventors have discovered that Picaridin and zinc ricinoleate can be used together and applied to clothing, skin, and other items. The present inventors have also discovered that when the two compounds are used together, the zinc ricinoleate surprisingly does not reduce the effectiveness of the Picaridin. In fact, the presence of the two compounds may synergistically improve in effectiveness when used together in at least some circumstances.

When used together, Picaridin and zinc ricinoleate may be particularly suitable for outdoor activities, like hiking, camping, hunting, and fishing. In these activities, it can be particularly helpful to provide insect repellence while also masking or eliminating both the odor of the repellent and human scent itself.

Both Picaridin and zinc ricinoleate would typically be used and applied in liquid, suspension, or solution form, and would be sprayed, spread, or otherwise coated on the surface or surfaces to be treated (e.g., clothing or equipment). The preparations of the compounds themselves may be standard commercial off-the-shelf preparations, e.g., 20% Picaridin solution and 0.5-2% zinc ricinoleate solution. These solutions may be applied to particular substrates in concentrations of, e.g., from about 1μL/cm² to about 10μL/cm², depending on the substrate, the situation, the desired effect, and other environmental factors.

The Picaridin and zinc ricinoleate may be supplied in separate solutions and applied separately, either at the same time or spaced in time. For example, in one embodiment, one would apply both compounds at the same time, while in another embodiment, one would spray one component on the substrate, wait several minutes until it has dried, and then spray the second component on the substrate. The compounds may be reapplied every few hours, e.g., every 4-6 hours, or when a decrease in efficacy is noted.

In some embodiments, it may be possible to provide both Picaridin and zinc ricinoleate in the same liquid carrier medium. However, the two compounds are soluble to different extents in different solvents, and for that reason, it may be easier and more expedient simply to use multiple solutions. The order in which the two compounds are applied may vary from embodiment to embodiment. In one embodiment, the Picaridin may be applied first, followed by the zinc ricinoleate, although that order may vary depending on the nature and composition of the solutions and other factors.

Embodiments of the invention include kits that include a spray bottle or mist sprayer of each compound in a solution of appropriate concentration, along with directions for applying the compounds. Some embodiments may use the two-package approach for zinc ricinoleate disclosed in U.S. application Ser. No. 13/360,369.

Although a number of embodiments may use liquid preparations of both compounds, in some embodiments, one component may be a liquid while another component may at least initially be in solid form. For example, when preparing clothing for hunting and other outdoor activities, the clothing may be washed with a fragrance-free detergent that includes a significant amount of zinc ricinoleate, for example, LETHAL® human scent eliminator laundry detergent (Clean Control Corporation, Warner Robins, Ga.). The Picaridin may then be applied to the wet or dried clothing. Similarly, a person may take a shower or bath using an unscented soap or body wash that contains significant amounts of zinc ricinoleate, such as LETHAL® human scent eliminator shampoo and body wash (Clean Control Corporation, Warner Robins, Ga.) and then apply Picaridin solution.

Other methods of preparing items for outdoor activities like hunting may involve spraying, soaking, or otherwise treating the item, or an area of the item, with one solution and then spraying, soaking, or otherwise treating the item, or an area of the item, with the other solution. As another example, solutions of appropriate concentrations may be supplied in the form of “body wipes,” i.e., with the solutions pre-soaked and absorbed into a disposable cloth and ready to be wiped onto skin or clothing. In some embodiments, it may be useful to allow the item to dry between applications. In other embodiments, it may be useful to allow the item to dry after both Picaridin and zinc ricinoleate have been applied.

The following examples serve to illustrate particular embodiments and aspects of the invention.

### EXAMPLES

In the following examples, the Picardin was in the form of a 20% by weight commercial, ready-to-use solution and LETHAL® Field Spray and Activator (both Clean Control Corporation, Warner Robins, Ga.) was used in its commercial, ready-to-use form as a source of zinc ricinoleate (U.S. patent application No. 13/360,369, which was incorporated by reference above).

### Example 1

### Spatial Repellency Testing

The impact of aqueous solutions of zinc ricinoleate on the behavioral effects on mosquitoes of 20% Picaridin solution was evaluated. Picardin and zinc ricinoleate solutions were selected as described above, and were tested by applying in two different amounts or concentrations, 7.9 μL/cm² and 1.6 μL/cm². Additionally, yellow fever mosquitoes, Aedes aegypti, were obtained from laboratory colonies at the Iowa State University, Medical Entomology Laboratory (Ames, Iowa).

A static-air choice-test apparatus was used to determine the behavior effects of the compounds on the insects. The apparatus comprised a 9×60 cm section of glass tubing with a 2 cm hole drilled at the midpoint along the length for central introduction of the insects. One side of the apparatus was treated, while the other was not. The position of the treated side, to the left or the right of the midpoint hole, was selected by using a random-number table.

Samples to be tested were pipetted directly onto filter paper (surface area 63.6 cm²) and allowed to sit at room temperature for 20 minutes prior to testing. Treated filter papers were then placed inside the lids of 9 cm glass Petri dishes for evaluation of spatial repellency.

For each test, approximately 20 adult female mosquitoes were immobilized by CO₂ and then introduced into the test apparatus through its central hole. Three replicates of each sample were completed. Timing began immediately following mosquito introduction and ended after 15 minutes. The results include observations of spatial repellency measured as a percentage repellency calculated by the formula of Equation (1) below:

[(Individuals on Untreated Side—Individuals on Treated Side)+20]*100 (1)

The results of the testing are set forth in Table 1 below.

TABLE 1
Results of Example 1.

| | | Average |
|---|---|---|
| | | Percentage |
| | Amount | Repellency |
| Treatment | Applied | 15 min. |
| 20% Picaridin Bug & Tick Repellent | 500 μl | 76.7% |
| 20% Picaridin Bug & Tick Repellent | 100 μl | 57.8% |
| 20% Picaridin Bug & Tick Repellent + | 500 μl + 500 μl | 95-100% |
| Zinc Ricinoleate | | |
| 20% Picaridin Bug & Tick Repellent + | 100 μl + 100 μl | 81.4% |
| Zinc Ricinoleate | | |
| Untreated Control | — | −13.2% |

Surfaces treated with 20% Picaridin and 20% Picaridin combined with zinc ricinoleate were similarly repellent (57-76% spatial repellency) to the adult yellow fever mosquito at the 1.6μL/cm² rate. At the higher rate of 7.9μL/cm², the 20% Picaridin combined with zinc ricinoleate caused fumigant effects. Mosquitoes were knocked down following 15 minutes of exposure (81% spatial repellency=100% knock down).

### Example 2

### Field Study

In order to confirm the results of Example 1, field studies were conducted with volunteers wearing short sleeves that left skin exposed. Volunteers were treated with Picaridin on both arms and zinc ricinoleate in addition to Picaridin on only the right arm. The test solutions were mixed in the laboratory, and volunteers were treated approximately 30 minutes before field exposure. Picaridin and zinc ricinoleate were sourced as described above and applied as follows:

20% Picaridin, ready-to-use formulation, applied neat. 1.0 g per arm of this solution was applied via 8 sprays of the formulation from a 2.0 oz mist sprayer.

1.0 g of zinc ricinoleate solution was applied to the right arm only via 1 spray from a 24 oz mist sprayer with trigger.

Untreated control subjects and subjects treated with repellent and repellent/zinc ricinoleate were exposed to a wooded area with observed mosquito populations in Perry, Ga. in mid-June. Treated subjects were partnered in groups of two, and each partner monitored the front of their own exposed forearms and necks and the back of their partner's forearms and neck. Mosquitos landing with intent to bite (LIIBe) were recorded. Testing began at 7PM (before dusk) and ended at 10PM (after dusk). Table 2 summarizes the results of Example 2.

TABLE 2
Results of Example 2.

| | Amount Applied | | | | | | | | | |
|---|---|---|---|---|---|---|---|---|---|---|
| | Left | Right | Number of Bites | | | | | | | |
| Treatment | Arm | Arm | L1 | L2 | L3 | L4 | R1 | R2 | R3 | R4 |
| 20% Picaridin | 1 | 1 | 0 | 0 | 0 | 0 | − | − | − | − |
| Bug & Tick | gram | gram | | | | | | | | |
| Repellent | | | | | | | | | | |
| 20% Picaridin | NA | 1 | − | − | − | − | 0 | 0 | 0 | 0 |
| Bug & Tick | | gram + | | | | | | | | |
| Repellent + | | 1 | | | | | | | | |
| Lethal Spray | | gram | | | | | | | | |

| | | F1 | F2 | F3 | F4 | N1 | N2 | N3 | N4 |
|---|---|---|---|---|---|---|---|---|---|
| Untreated | Control | 4 | 3 | 5 | 6 | 2 | 4 | 3 | 2 |
| Control | | | | | | | | | |
| (Face | | | | | | | | | |
| and Neck) | | | | | | | | | |

Participants were also asked to gauge the odor(s), if any, emanating from each arm. The results of this odor study are shown in Table 3.

TABLE 3
Results of Odor Study.

| | Amount Applied | | | | | | | | | |
|---|---|---|---|---|---|---|---|---|---|---|
| | Left | Right | Odor | | | | | | | |
| Treatment | Arm | Arm | L1 | L2 | L3 | L4 | R1 | R2 | R3 | R4 |
| 20% Picaridin | 1 | 1 | + | + | + | + | − | − | − | − |
| Bug & Tick | gram | gram | | | | | | | | |
| Repellent | | | | | | | | | | |
| 20% Picaridin | NA | 1 | − | − | − | − | 0 | 0 | 0 | 0 |
| Bug & Tick | | gram + | | | | | | | | |
| Repellent + | | 1 | | | | | | | | |
| Lethal Spray | | gram | | | | | | | | |

The results of Example 2 confirm the laboratory results of Example 1: the repellent effects of Picaridin are not retarded or negatively impacted by the presence of zinc ricinoleate. Moreover, the volunteers perceived no significant odors from their right arms, which had received zinc ricinoleate in addition to Picaridin. This indicates that zinc ricinoleate is effective against the odor of Picaridin itself

While the invention has been described with respect to certain embodiments, the embodiments are intended to be exemplary, rather than limiting. Modifications and changes may be made within the scope of the invention, which is defined by the appended claims.

What is claimed is:

1. A method for reducing the perceptible odor of a Picaridin insect repellent composition, comprising:
applying the Picaridin insect repellent composition to an area of a substrate; and
applying zinc ricinoleate to the area of the substrate.

2. The method of claim 1, wherein the Picaridin insect repellent composition and the zinc ricinoleate are in separate suspensions or solutions.

3. The method of claim 2, wherein the Picaridin insect repellent composition suspension or solution has a concentration of Picaridin of about 20%.

4. The method of claim 2, wherein the zinc ricinoleate suspension or solution has a concentration of zinc ricinoleate of about 0.5-2%.

5. The method of claim 2, wherein the separate suspensions or solutions are each applied in an amount of about 1-10μL/cm².

6. The method of claim 5, wherein the separate suspensions or solutions are each applied in an amount of about 1.6μL/cm².

7. The method of claim 2, further comprising allowing one of the separate solutions or suspensions to dry on the area of the substrate before applying the other of the separate solutions or suspensions.

8. A method of preparing clothing for an outdoor activity, comprising:
washing the clothing using an odor-suppressing detergent including zinc ricinoleate in an effective amount; and
applying a solution including Picaridin in an effective amount to the clothing.

9. The method of claim 8, further comprising drying the clothing before said applying.

10. The method of claim 8, wherein the outdoor activity comprises hunting.
